(19) United States
(12) Patent Application Publication
(10) Pub. No.: US 2012/0303040 A1
(43) Pub. Date: Nov. 29, 2012

(54) Circumcision Device and Procedure

(75) Inventors: R. Michael Johnson, York, PA (US); Pietro Ranieri, Haddonfield, NJ (US); Kristin Chrouser, Baltimore, MD (US); Sheila Green, Dillsburg, PA (US)

(73) Assignee: JHPIEGO CORPORATION, Baltimore, MD (US)

(21) Appl. No.: 13/114,692

(22) Filed: May 24, 2011

(51) Int. Cl. A61B 17/326

(52) U.S. Cl. 606/118

(57) ABSTRACT

A male circumcision device which reasonably skilled and trained caregivers in emerging nations can use to perform painless, safe, effective, quick and minimal cost circumcisions includes: (a) an enclosure with an opening configured to allow it to slide, after the penile foreskin has been rolled back, over the glans of the patient's penis until this opening is located adjacent the point where the foreskin attaches to the penis, the enclosure further having a plurality of cavities which are arranged around its perimeter and proximate its opening, (b) male parts of a clamping assembly, each being configured to be detachably fitted into one of the enclosure's cavities and each having an elongated protuberance with a distal end that can pierce the penile foreskin, and (c) female parts of a clamping assembly, each having an opening to receive the protuberances' distal ends and each having a configuration adapted to allow these male and female parts to mate together so as to apply a compressive force between them that aids in holding a patient's remaining foreskin together while it heals.

### BACKGROUND OF THE INVENTION

1. Field of the Invention

The present invention relates generally to surgery devices and methods. More particularly, the present invention relates to a circumcision device and procedure.

2. Description of the Related Art

Circumcision is a well-established surgical procedure that has been practiced for centuries for medical needs or religious purposes. The procedure involves the use of scissors and clamps to cut the penile foreskin and the subsequent use of suture materials to approximate the cut ends of the foreskin. In the modern operating rooms of developed countries, it is a relatively brief and inexpensive surgical procedure for which an assortment of surgical devices have been developed. See U.S. Pat. Nos. 4,491,132, 5,797,921, 5,860,988, 7,303,567, 7,591,824 and U.S. Patent Application Publication No. 2006/0219753.

However, in emerging nations where access to such modern operating rooms often doesn't exist, providing for the availability of large numbers of painless, safe, and effective circumcisions is a seemingly insurmountable task. In those nations whose populations are being ravaged by HIV/AIDS, the inability to provide large numbers of painless, safe, and effective circumcisions can be a life or death matter since various health studies have shown that removing the foreskin dramatically impedes the spread of HIV/AIDS. Some attempts have been made to develop specialized surgical devices for use where fully qualified medical practitioners and facilities are often not readily available. See U.S. Pat. Nos. 5,269,788, 5,649,933, U.S. Patent Application Publication No. 2008/0154283 and PCT Patent Publication WO2011/007358A2.

The objective of the present invention is to provide a means to perform a male circumcision in an emerging nation, where fully qualified medical practitioners and facilities are often not readily available. The circumcision device of the present invention has been designed for use by caregivers who can be relatively easily taught how to use it and perform the novel steps involved in the circumcision procedure of the present invention.

### SUMMARY OF THE INVENTION

Recognizing the need for the development of improved circumcision methods and devices that can be used in emerging nations, the present invention is generally directed to satisfying this need and overcoming barriers to circumcising large percentages of the adult, male populations of emerging nations.

In accordance with a preferred embodiment of the present invention, a male circumcision device which reasonably skilled and trained caregivers in emerging nations can use to remove a patient's penile foreskin painlessly, safely, effectively, quickly and for a minimal cost includes: (a) a tubular enclosure or glans cap with an opening at its proximal end that allows it to slide over the glans of the patient's penis until this opening is adjacent the point where the patient's foreskin attaches to the penis, and further configured with an outer surface having an interaction portion that includes a plurality of cavities, (b) a plurality of male parts of a clamping assembly, each of these male parts having an outer and an inner surface, wherein from each outer surface there extends an elongated protuberance with a free, distal end that is configured to pierce a patient's penile foreskin, and wherein each inner surface is configured to allow one of these male parts to be detachably fitted into each of the enclosure's cavities, and (c) female parts of this clamping assembly, each having an inner surface with an opening and a configuration that is adapted to allow these females part to be affixed onto and around the perimeter of the interaction portion of the enclosure's outer surface in such a manner that each the opening aligns with the free ends of the male parts' protuberances when these male parts are themselves situated or detachably fitted into the enclosure's cavities, and wherein these female part inner surfaces and the male parts' outer surfaces are configured to have complimentary curvature which allows these surfaces to be brought together to apply a relatively uniform compressive force to the patient's foreskin that is held between them.

This preferred embodiment may further include: (d) a compression ring having a slit whose opposing edges each contain part of a ratcheting mechanism that allows this ring's inner diameter to be adjusted through a range of values that includes a diameter of magnitude such that this compression ring can be fitted around the enclosure and clamped down upon it with enough compressive force so as to cut off the circulation to that part of a patient's foreskin which is compressed between the enclosure and this compression ring.

This preferred embodiment may also further include: (e) a tubular, shaft protector having a slit extending between its ends which allows this protector's internal diameter to be adjusted through a range of values.

Thus, there has been summarized above (rather broadly and understanding that there are other preferred embodiments which have not been summarized above) the present invention in order that the detailed description that follows may be better understood and appreciated.

### BRIEF DESCRIPTION OF THE DRAWINGS

FIG. 1 is a perspective view of a preferred embodiment of the present invention.

FIG. 2 is a perspective view of a preferred embodiment of the glans cap or enclosure of the present invention.

FIG. 3 is a perspective view of a preferred embodiment of a female part of the present invention and its union with three male parts of the clamping assembly of the present invention.

FIG. 4 is a side view of a preferred embodiment of the biodegradable male staple or elongated protuberance and its alignment block of the male part of the clamping assembly of present invention.

FIG. 5 is a cross-sectional view of the union of preferred embodiments for the female and male parts of the clamping assembly of present invention.

FIG. 6 is a cross-sectional view of an alternative embodiment of the male part of the present invention in the form of a needle and biodegradable suture material.

FIG. 7 shows a perspective view of another possible version of the male part of the clamping assembly of the present invention.

### DESCRIPTION OF THE PREFERRED EMBODIMENT

Before explaining at least one embodiment of the present invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The current invention includes both a circumcision device and the novel circumcision method or procedure that is made possible by this device. We begin our disclosure of the present invention by describing it as a male circumcision method which reasonably skilled caregivers (i.e., when fully qualified medical practitioners are not available) can perform to fully remove of a patient's foreskin in a manner that is done painlessly, safely, effectively, quickly, and for a minimal cost.

The caregiver must thoroughly wash their hands with a disinfectant liquid; and then preferably put on surgical gloves. The patient is requested to undress and lie supine on a flat surface such as a bed or table. A standard surgical prep solution is used to cleanse the entire penis, including both the inner and outer foreskin surfaces.

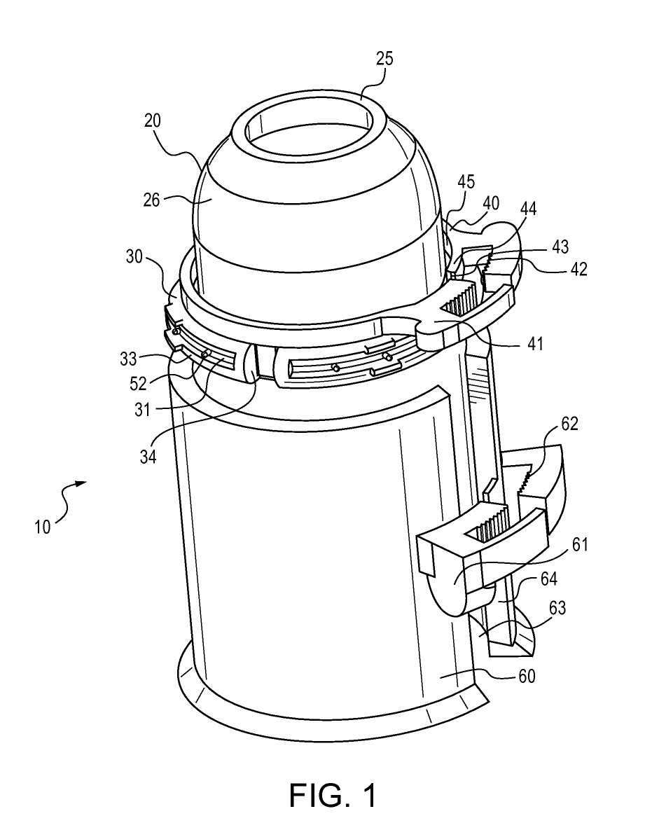

As a means to support the present invention's shaft protector 60, a large flat support plate with a hole in the center is slid over the penis, and laid flat upon the patient's torso. Referring to FIG. 1, the shaft protector is slid down over the shaft of the penis, and is allowed to rest its base upon the support plate. The shaft protector 60 is then snugly tightened to provide an upright support to the penis during the procedure. The shaft protector thumb grip 61 and the shaft protector block ratchet mechanism 62 are used to allow the shaft protector to be snugly fastened around the shaft and to allow the shaft protector to stay in place until the procedure has been fully completed. It is anticipated that there will be multiple sizes available of the present invention's circumcision device 10 from which the caregiver would select the size which is appropriate to the patent being treated.

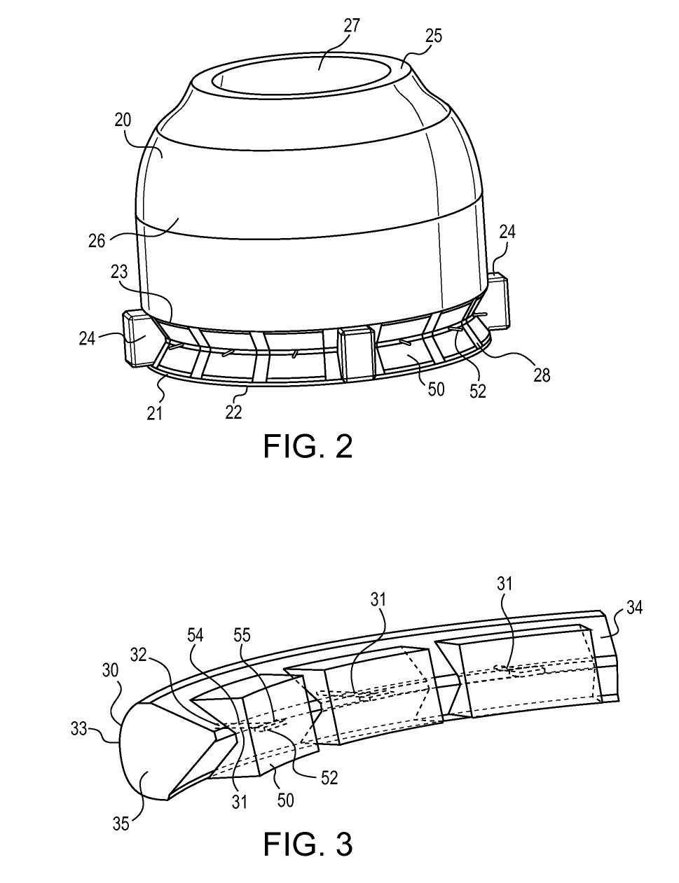

A circular, tubular-like, inner ring or glans cap or protective enclosure 20, as seen in FIG. 2, has an opening 21 in its proximal end 22 that slides down, after a patient's foreskin has been rolled back or retracted, over the glans of the patient's penis until the enclosure's proximal end bottoms out at the inside of the foreskin at the point where the foreskin attaches to the penis. The foreskin is then reduced or slid back up over the proximal end of the enclosure so that it lies adjacent to the enclosure's outer surface 26.

This enclosure is especially configured near its proximal end 22 to have an interaction portion 28 that includes twelve cavities or compartments 23, which are separated by a number of guide blocks or positioning ribs 24. Each of these cavities holds the pre-installed and detachable, inner or male part of a clamping assembly that consists of a staple cap or base 50 and its contoured outer face 51 from which extends a biodegradable male staple 52.

A circular outer ring or compression or crushing ring 40 is then slid down over the outside of the foreskin until the compression ring rests above the enclosure's guide blocks 24 and this compression ring is clamped down onto the enclosure so as to compress the foreskin and cut the circulation off in the foreskin by using this ring's thumb grip 41 and block ratchet mechanism 42. In this way, blood flow to the upper part of the foreskin is interrupted, which will minimize the amount of blood loss when the incision is subsequently performed. In addition, that portion of the foreskin which is crushed will die and eventually be sloughed off, thus minimizing the amount of foreskin that remains attached to the penis.

The outer or female part of the clamping assembly consists of four female staples or staple holders 30 that are inserted outside the foreskin and into the slots or spaces between the guide blocks which the lower portion of the outer surface of the enclosure has been configured to provide; see FIG. 2. The enclosure's guide blocks or positioning ribs 24 serve to show where to position each of the four female staple holders 30. Each of these female staple holders connects with the ends of three of the biodegradable male staples 52 and enough manual force is applied to these female staple holders until the piercing points 53 on the male staples penetrate through the foreskin, and then this application of force or pushing is continued until each of the female staple holders become fully mated with its matching combination of three biodegradable male staples 52.

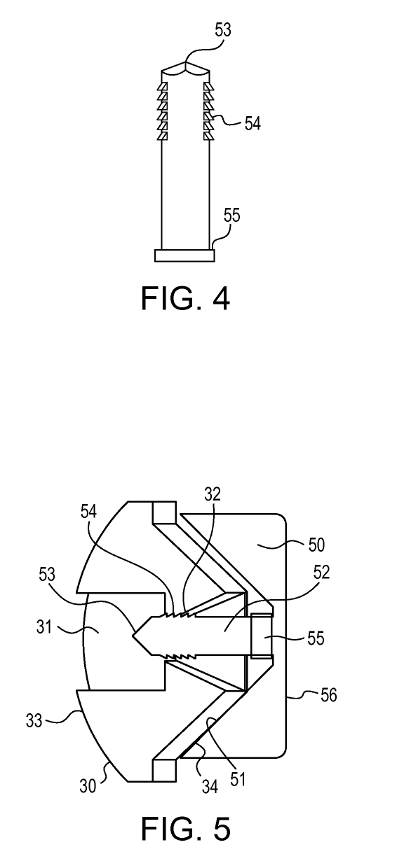

As seen in FIG. 4, the shafts of these biodegradable male staples 52 are designed so that a portion of them have a ratchet male mechanism 54 (e.g., the staple's outer surface having a saw-tooth shape over a portion of the staple's surface). The purpose of this is to connect and lock into a trough-like opening 31 in the female staple holder and to allow for patient-specific compression (since foreskin thickness varies from patient to patient).

A suitable cutting means is then used to make the circumferential cut around the outer surface of the foreskin. This cut is made by guiding the cutting means along the top edge of the compression ring. The glans of the penis is protected during this incision, since the glans is fully covered by the enclosure 20 which was fitted over the glans and has the foreskin resting against the enclosure's outer surface 26.

To remove this circumcision device 10, the following procedure is used. The compression ring 40 is loosened by releasing the ring's thumb grip and block ratchet mechanism. The compression ring can then be lifted off.

To remove the enclosure 20 it is first necessary to loosen the twelve staple bases 50 from their detachable and frictional fit inside the cavities 23 in the interaction portion of the enclosure. This is accomplished by gently pulling on each of the four female staple holders 30 until the staple caps come loose from the enclosure. It is now possible to slip the enclosure cap up and off of the glans.

The shaft protector 60 is now loosened by unfastening its thumb grip and block ratchet mechanism. The shaft protector and the support plate can now be slid off of the penis.

Remaining on the patient are the four clamping assemblies which are arrayed around the circumference of the penis. Since the male staples 52 are biodegradable, when these staples disintegrate after several days, the other parts of the clamping assemblies will fall off and can be discarded by the patient. During this disintegration period, the compressive action due to the connection of the staple cap 50 to the female staple holders 30 assures that the remaining skin of the foreskin will stay tightly held together during the healing process.

Turning our attention now to the various components which comprise the present invention, it is seen that the present invention's enclosure 20 is a tubular structure with inner 27 and outer 26 surfaces, proximal 22 and distal 25 ends and an especially configured concaved, interaction portion 28 of its outer surface that is located near its proximal end and that has around its perimeter twelve, relatively uniformly distributed cavities 23 whose dimensions are chosen so as to provide a frictional fit with the inner or male parts 50 of the clamping assemblies that are pre-installed and detachably fitted into these cavities. Around this perimeter are also distributed four guide blocks or positioning ribs 24 that, together with the surface concavity in this area, are used to help properly fit and located the four female/outer parts 30 of the present invention's clamping assembly.

This enclosure 20 is designed to perform several functions. First, it must fit snugly over the glans and be able to slide all the way inside the foreskin until it reaches the bottom of the space between the glans and the foreskin. In this way, the enclosure is able to fully encase and protect the glans from all the steps involved in removing a patient's foreskin. At the step in the present invention's circumcision procedure when the compression ring 40 is slid down over the foreskin and is then tightened so as to provide a compressive force upon the foreskin, it is the enclosure 20 which now provides a back-up force for this compressive action. That is, the foreskin is compressed between the enclosure and compression ring.

At the step when the incision is made to remove the foreskin, here again—the enclosure provides a back-up to insure that the cutting means is only able to cut through the foreskin and not harm the fully protected glans.

Since this circumcision procedure is to be done on a flaccid penis, the shaft protector 60 is used to cause the penis to maintain an upright position to make it easier for the caregiver to perform each step in the procedure. The shaft protector is seen to be an adjustable diameter, tubular device which has a slit 63 which extends between its two ends that allows for it diameter adjustability through a range of values. On each edge 64 of this slit is a thumb grip 61 and a block ratchet 62 which comprise an adjustable ratcheting mechanism whose parts can be brought together in such a way so as to incrementally set the gap distance between this slit's edges 64 and thereby adjustably set the inner diameter of the protector. Here again, several sizes of the shaft protector 60 are available for the caregiver to choose among. The easy-to-use ratcheting or latching mechanism allows the caregiver to fasten the shaft protector firmly around the shaft of the penis, but without causing discomfort.

As previously mentioned, an additional role for the enclosure 20 is to provide support and alignment for the inner or male parts of the present invention's clamping assembly, i.e., the twelve biodegradable male staples or elongated protuberances 52 which are affixed on their proximal ends to the twelve staple caps or bases 50. As seen in FIGS. 4-5, these staple bases have an inner 56 and an outer 51 surface and a centrally located opening in this outer surface in which the proximal end or alignment block 55 of the male staple 52 is fitted. This outer surface 51 is especially configured to align and mate with the inner surface 34 of the female staple holder 30 (e.g., these surfaces 51, 34 have complimentary curvature) so that these surfaces can be brought together over most of, or at least a large extent of, their surfaces areas to apply a relatively uniform compressive force to the foreskin held between them when a radial inwardly directly force is applied to the outer surface 33 of a female staple holder 30. The purpose of this compressive action is to assure that a patient's remaining foreskin will stay tightly held together until it heals.

These staple bases 50 are fitted with a very slight frictional fit into the enclosure's cavities so that they can easily be pulled free at the conclusion of the circumcision. In this way, the twelve male staples are accurately pre-aligned with the trough-like opening in each of the female staple holders. To complete this alignment, the four female staple holders 30 are guided into position by the enclosure's four guide blocks 24.

The outer parts of the present invention's clamping assembly are its female staple holders 30. As shown in FIGS. 3 and 5, each of the present invention's four female staple holders is an approximate one-forth portion of a circular ring-shaped structure having a convex inner surface 34 and a rounded outer surface 33 and two ends 35. Designed into each of the four female staple holders is a trough-like opening 31 which is sized to accept and fit with the distal ends of three biodegradable male staples 52. The female staple holders are designed to fit within the space that is defined by pairs of the enclosure's guide blocks 24 so that the opening in the female staple holders will accurately align with the twelve male staples 52. Furthermore, as noted above, the outer surface of the staple bases 50 are designed to be concave, so that they will nest with the convex inner surface of the female staple holders thus further assuring that the twelve male staples will accurately align with the trough-like openings in the female staple holders. Each of the trough-like openings 31 in the female staple holders extends between a holder's inner and outer surfaces and each of these opening's have a ratchet female mechanism 32 to facilitate the locking into place with the matching ratchet male mechanisms 54 that is located on the male staple's outer surface proximate its distal end or piercing point 53.

The compression ring 40 has a slit 43 and opposing slit edges 44 which allows the diameter of the ring's inner surface 45 to be adjusted. Thumb grips 41 and block racheting mechanisms 42 are located on the slit's opposing edge and these interact to provide for the enclosure's diameter adjustability through a range of values. This compression ring is configured such that its nominal inside diameter is sufficient to allow it to easily fit over and around the enclosure 20. Once the compression ring is in place over the foreskin, it is tightened, using its thumb grip and block ratchet mechanism, until it produces a crushing compression upon the foreskin. The upper edge of the compression ring is then used as a guide to make the circumcision cut all around the circumference of the foreskin.

Several alternative cutting means can be used to make the circumcision cut around the circumference of the foreskin. Among these are a scalpel, a rotary cutting instrument (similar to a pizza cutter), a cutter of the configuration as used to cut the foil on a wine bottle, or a garrote (thin wire).

The foregoing is considered as illustrative only of the principles of the invention. For a first example, the present invention could just as well been described with an alternative clamping assembly.

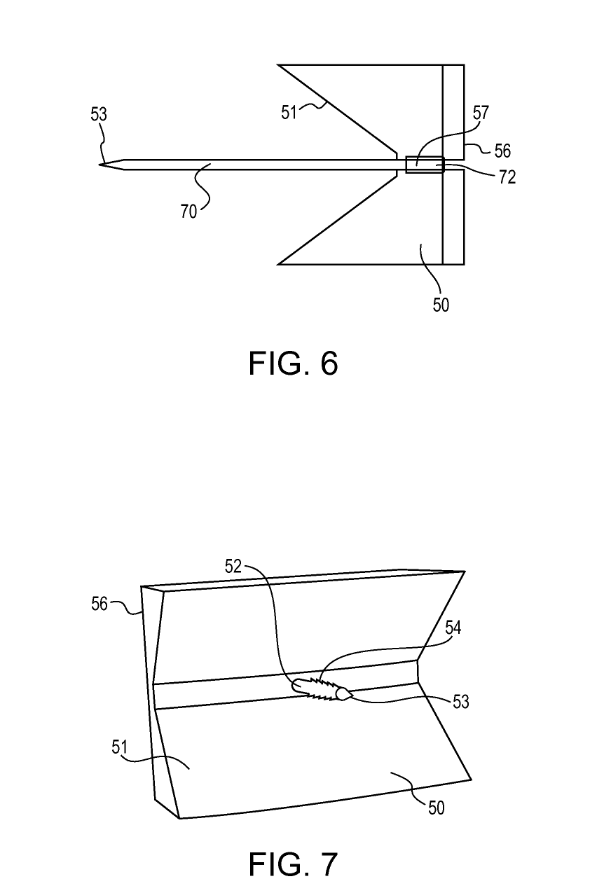

Instead of using the rigid, biodegradable male staples 52, the present invention can utilize segments of biodegradable suture material which are affixed to surgical needles whose lengths are such that they extend all the way through the female side 30 of the clamping assembly; see FIG. 6. This alternative clamping assembly has the advantage of a further reduction in the cost of the present invention, since the material that is needed to fabricate the male staples 52 (i.e., a combination of L-lactide and glycolide polymers) is very expensive even though only a small amount is needed for each male staple.

In this alternative clamping assembly, a small surgical needle 70 with the suture 72 affixed, is held within each of inner parts 50 of this clamping assembly. When the four outer or female parts 30 are pressed into place, the needles 70 will pierce through the foreskin and extend out the back side of the outer part 30. The caregiver then grasps the needle 70 and pulls it all the way out, thus pulling the suture 72 out of its storage space 57 in the inner part and all the way though and out of the back of the outer part. The caregiver then pulls firmly on the free end of the suture, until the outer part is tightly clamped onto the foreskin. Then the caregiver secures the suture tightly against the back side of the outer part 30 in the opening 31 using a suitable means such as a crimping tool and soft metal crimp. Other means may also be used to secure this end of the suture tightly to the outer part. Finally, the caregiver uses a knife or a scissors or other cutting means to snip off the excess amount of the suture and discards it along with the needle.

Whether one uses the staple version or the surgical needle with the suture affixed version, the patient, after all the other elements of the circumcision device have been removed, is left only with the four female or outer parts 30 and the twelve small male or inner parts 50 of the clamping assembly fastened to the remaining small portion of the foreskin. When the staple or suture biodegrades, these remaining parts of the circumcision device will fall off and can be discarded by the patient. The residual crushed portion of the foreskin will also be sloughed off, because it has ceased to be viable due to the crushing action of the compression ring.

For a second example, the present invention could just as well been described with an alternative staple base 50. As shown in FIG. 7, an alternative design for this base would be a simplification which would eliminate its alignment block 55 and thus have the proximal end of the male staple 52 connected directly into the base.

After much experimentation, it was found that suitable materials from which to fabricate the present invention's biodegradable staple appear to be a combination of L-lactide and glycolide polymers, with the ratio having a preferred value of approximately 1/9. This preferred combination is generally equivalent to PURASORB® PLG 1017, which is manufactured by Purac Biomaterials, Gorinchem, The Netherlands. The other parts of the present invention are made from a PF 511 FDA polypropylene material. Suitable biodegradable suture materials are well known in the art and therefore will not be discussed herein.

Since numerous modifications and changes will readily occur to those skilled in the art, it is not desired to limit the invention to the exact construction and operation shown and described herein. Accordingly, all suitable modifications and equivalents may be resorted to, falling within the scope of the invention that is hereinafter set forth in the claims to the invention.

We claim:

1. A circumcision device that allows for the removal of the penile foreskin of a patient at approximately the point where said foreskin attaches to the penis and without damage to the shaft and glans of the penis of said patient, said device comprising:
an enclosure having a perimeter and a proximal end that has an opening, said opening having a configuration adapted to allow said opening to slide over the glans of the penis until said opening is located directly adjacent the point where the foreskin attaches to the penis, said enclosure further having a plurality of cavities which are arranged around the perimeter of said enclosure proximate said opening,
a clamping assembly having male and female parts,
wherein each of said male parts having an outer surface from which extends an elongated protuberance with a distal end having a configuration that is adapted to pierce the penile foreskin of a patient, and each of said male parts having a configuration adapted to allow said male parts to be detachably fitted into one of said cavities of said enclosure, and
wherein each of said female parts having an inner surface which includes an opening to receive said protuberance distal ends of said male parts, and each said inner surface of said female parts having a configuration adapted to allow said female part inner surfaces to mate with said male part outer surfaces so as to apply a compressive force between said inner and outer surfaces to aid in holding a patient's remaining foreskin together while it heals from said removal of penile foreskin.

2. The circumcision device as recited in claim 1, further comprising:
a compression ring having an inner surface with a diameter, a slit with opposing slit edges, and a ratcheting mechanism having a configuration adapted to allow said inner surface diameter to be adjusted through a range of values that include those necessary to allow said compression ring to fit around said enclosure and for said inner surface diameter to be then reduced in value until said compression ring and said enclosure act together to apply a compressive force of sufficient magnitude so as to minimize the circulation in any penile foreskin between said compression ring and said enclosure.

3. The circumcision device as recited in claim 1, wherein:
said male part elongated protuberance chosen from the group consisting of a staple and a needle.

4. The circumcision device as recited in claim 2, wherein:
said male part elongated protuberance chosen from the group consisting of a staple and a needle.

5. The circumcision device as recited in claim 3, wherein:
said needle having attached thereto a suture, and
said staple having a portion with a configuration adapted to allow said staple distal end to be locked into said opening of said female part of said clamping assembly.

6. The circumcision device as recited in claim 4, wherein:
said needle having attached thereto a suture, and
said staple having a portion with a configuration adapted to allow said staple distal end to be locked into said opening of said female part of said clamping assembly.

7. The circumcision device as recited in claim 5, wherein:
said staple and said suture being fabricated from biodegradable materials.

8. The circumcision device as recited in claim 6, wherein:
said staple and said suture being fabricated from biodegradable materials.

9. The circumcision device as recited in claim 1, further comprising:
a tubular protector having a configuration adapted to protect the shaft of the penis during the removal of the penile foreskin.

10. The circumcision device as recited in claim 2, further comprising:
a tubular protector configured to protect the shaft of said penis during said removal of said penile foreskin.

11. The circumcision device as recited in claim 3, further comprising:
a tubular protector configured to protect the shaft of said penis during said removal of said penile foreskin.

12. The circumcision device as recited in claim 4, further comprising:
a tubular protector configured to protect the shaft of said penis during said removal of said penile foreskin.

13. The circumcision device as recited in claim 5, further comprising:
a tubular protector configured to protect the shaft of said penis during said removal of said penile foreskin.

14. The circumcision device as recited in claim 6, further comprising:
a tubular protector configured to protect the shaft of said penis during said removal of said penile foreskin.

15. The circumcision device as recited in claim 7, further comprising:
a tubular protector configured to protect the shaft of said penis during said removal of said penile foreskin.

16. The circumcision device as recited in claim 8, further comprising:
a tubular protector configured to protect the shaft of said penis during said removal of said penile foreskin.

17. Use of the circumcision device according to any one of claims 1 to 16 to allow for the removal of the penile foreskin of a patient at approximately the point where said foreskin attaches to the penis and without damage to the shaft and glans of the penis of said patient.
